(19) United States
(12) Patent Application Publication
(10) Pub. No.: US 2015/0164794 A1
(43) Pub. Date: Jun. 18, 2015

(54) METHOD FOR PREPARING AQUEOUS NANOPARTICLE SUSPENSIONS OF DERIVATIVES OF 4,9-DIHYDROXY-NAPHTHO[2,3-b]FURAN ALIPHATIC ACID ESTERS

(71) Applicants: Zhiwei Jianh, Stow, MA (US); Xian Li, Fengcheng (CN); Hairong Xu, Jiaojiang (CN); Yan Ye, Zhoushan (CN); Hongwei Hu, Suifenhe (CN); Huanhuan Yang, Qianyuan Town (CN)

(72) Inventors: Zhiwei Jianh, Stow, MA (US); Xian Li, Fengcheng (CN); Hairong Xu, Jiaojiang (CN); Yan Ye, Zhoushan (CN); Hongwei Hu, Suifenhe (CN); Huanhuan Yang, Qianyuan Town (CN)

(73) Assignee: Zhoushan Haizhongzhou Xinsheng Pharmaceuticals, Co., Ltd., Zhoushan, Zhejiang (CN)

(21) Appl. No.: 14/379,247

(22) PCT Filed: Feb. 17, 2012
(86) PCT No.: PCT/CN2012/000190

§ 371 Date: Feb. 25, 2015

(51) Int. Cl. A61K 9/10; A61K 31/343

(52) U.S. Cl. CPC A61K 9/10; A61K 31/343

(57) ABSTRACT

Disclosed is a method for preparing aqueous nanoparticle suspensions of derivatives of 4,9-dihydroxy-naphtho[2,3-b]furan aliphatic acid esters. The compositions and the uses of the aqueous nanoparticle suspensions prepared according to the method as described herein are also disclosed.

### CROSS-REFERENCE TO RELATED APPLICATIONS

This application is the U.S. national stage application of International Patent Application No. PCT/CN2012/000190, filed Feb. 17, 2012, the entire contents of which are incorporated herein by reference.

### TECHNICAL FIELD

The present application relates to a method for preparing aqueous nanoparticle suspensions of derivatives of 4,9-dihydroxy-naphtho[2,3-b]furan aliphatic acid esters. The present application also relates to the compositions of the aqueous nanoparticle suspensions prepared according to the method as described herein. The present application further relates to uses of the aqueous nanoparticle suspensions prepared according to the method as described herein.

### BACKGROUND

There is a critical need in the pharmaceutical industries to formulate a poorly water-soluble pharmaceutically active compounds into formulations suitable for oral, injectable, and other routes of delivery. Nanoparticle formulations containing the poorly water-soluble pharmaceutically active compounds provide advantages such as improved oral bioavailability, favorable toxicity profiles of the injectable formulations (e.g., due to the reduced use of organic solvents), passive targeting of certain cancerous tumors associated with loose fenestrated vasculature across which small drug particles can directly migrate, as well as sustained release forms of intramuscular injectable drugs.

As disclosed in our previous PCT application (CN2011/000357), therapeutic activities of 4,9-dihydroxy-naphtho[2,3-b]furans are mainly attributing to their ability to induce reactive oxygen species (ROS). Their prodrugs, derivatives of 4,9-dihydroxy-naphtho[2,3-b]furan esters, not only have improved stability in pharmaceutical compositions over the parent drug, but also reduced toxicity compared to the parent drug by avoiding unnecessary exposure to unintended target tissues.

However, some derivatives of 4,9-dihydroxy-naphtho[2,3-b]furan esters with broad therapeutic applications are poorly water-soluble, which make them difficult to formulate. So there remains a need to develop formulations of derivatives of 4,9-dihydroxy-naphtho[2,3-b]furan esters for their uses in the treatment of various diseases, disorders, and conditions.

### SUMMARY

A derivative of 4,9-dihydroxy-naphtho[2,3-b]furan aliphatic acid ester is as shown in formula I:

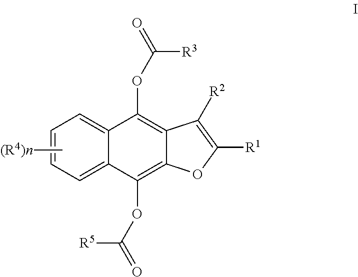

or a pharmaceutically acceptable salt thereof;

wherein each of n, R¹, R², R³, R⁴, and R⁵ is as defined and described herein.

In one aspect, the present invention provides a method for preparing an aqueous nanoparticle suspension of a compound of formula I. The method comprises: (1) dissolving a compound of formula I and optionally pharmaceutically acceptable surfactant(s) in a water-miscible organic solvent to form an organic solution; (2) dissolving optionally pharmaceutically acceptable agent(s) and/or optionally pharmaceutically acceptable surfactant(s) in water to form an aqueous solution; and (3) mixing the organic solution and the aqueous solution to form a nanoparticle suspension.

In another aspect, the present invention provides an aqueous nanoparticle suspension of a compound of formula I, prepared according to the method as described herein.

In yet another aspect, the present invention provides a kit which can be used for preparing an aqueous nanoparticle suspension of a compound of formula I. The kit comprises: (1) an organic solution of a compound of formula I and optionally pharmaceutically acceptable surfactant(s) in a water-miscible organic solvent, or components for preparing such solution; (2) an aqueous solution of optionally pharmaceutically acceptable agent(s) and/or optionally pharmaceutically acceptable surfactant(s) in water, or components for preparing such solution; and (3) instruction on how to mix the organic solution and the aqueous solution to form a nanoparticle suspension and also on how to prepare the organic solution and/or the aqueous solution if components instead of finished solution(s) are provided in the kit.

In a further aspect, the present invention provides a pharmaceutical composition comprising an aqueous nanoparticle suspension prepared according to the method as described herein and optionally pharmaceutically acceptable carrier(s).

In another aspect, the present invention provides an aqueous nanoparticle suspension comprising:
about 0.1-20 mg/ml of a compound of formula I;
about 0-200 mg/ml of pharmaceutically acceptable agent(s);
about 0-200 mg/ml of pharmaceutically acceptable surfactant(s); and
about 0.1-50% by volume of water-miscible organic solvent or solvent mixture.

In yet another aspect, the present invention provides an aqueous nanoparticle suspension comprising:
about 0.1-20 mg/ml of 2-acetyl-4,9-dihydroxy-naphtho[2,3-b]furan aliphatic acid ester;
about 0-200 mg/ml of pharmaceutically acceptable agent(s);
about 0-200 mg/ml of pharmaceutically acceptable surfactant(s); and
about 0.1-50% by volume of water-miscible organic solvent or solvent mixture.

### BRIEF DESCRIPTION OF THE DRAWINGS

FIG. 1 shows particle distributions of nanoparticle suspension NS-VIII-4.00 which was prepared according to the method as described in example 12.

FIG. 2 shows time courses of concentrations of compound I, oxidized drug, and oxidized active metabolite in mouse plasma in the pharmacokinetic study of nanoparticle suspension NS-I-4 in ICR mice as described in example 13. 2A, time course of concentrations of compound I; 2B, time course of concentrations of 2-acetyl-naphtho[2,3-b]furan-4,9-dione (oxidized drug as shown in scheme 1); 2C, time course of concentrations of 2-(1-hydroxyl)ethyl-naphtho[2,3-b]furan-4,9-dione (oxidized active metabolite as shown in scheme 1).

FIG. 3 shows results in compound I anticancer efficacy study on nude mouse HCT116 tumor xenograft model. 3A, tumor volume vs treatment time in three different dosage arms; 3B, picture of isolated tumors from the tumor borne nude mice in three different dosage arms.

### DEFINITIONS

As used herein, the following definitions shall apply unless otherwise indicated.

The term “aliphatic” or “aliphatic group”, as used herein, means a straight (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule.

The term “aliphatic acid” or “aliphatic carboxylic acid”, as used herein, means a carboxylic acid with an aliphatic group.

The term “water” used herein means pure water, e.g., ionized water.

The term “aqueous solution” includes, but is not limited to, water, saline solution, dextrose solution, aqueous solutions listed above containing one or more pharmaceutically acceptable agent(s), and aqueous solutions listed above containing one or more pharmaceutically acceptable surfactant(s).

The term “D10”, “D50”, and “D90” refer to the particle diameter at which the cumulative volume of the finer particles reaches 10%, 50%, and 90% of the total volume of all particles, respectively.

As used herein and in the claims, the singular forms “a”, “an”, and “the” include the plural reference unless the context clearly indicates otherwise. Thus, for example, a reference to “a compound” includes a plurality of such compounds.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “prodrug” means an agent that is converted into the parent drug in vivo. In certain embodiments, a prodrug is easier to administer than a parent drug. In certain embodiments, a prodrug may also have improved stability in pharmaceutical compositions over the parent drug. In certain embodiments, a prodrug has reduced toxicity compared to the parent drug by avoiding unnecessary exposure to unintended target tissues.

The terms “administer,” “administering,” or “administration”, as used herein, refer to either directly administering a compound or composition to a patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration”, “administered systemically”, “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “palliative” refers to treatment that is focused on the relief of symptoms of a disease and/or side effects of a therapeutic regimen, but is not curative.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition for the purpose of decreasing the risk of developing pathology associated with the disease, disorder, and/or condition.

The expression “unit dose” as used herein refers to a physically discrete unit of a formulation appropriate for a subject to be treated. It will be understood, however, that the total daily usage of a formulation of the present invention will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. A particular unit dose may or may not contain a therapeutically effective amount of a therapeutic agent.

An individual who is “suffering from” a disease, disorder, and/or condition has been diagnosed with and/or displays one or more symptoms of the disease, disorder, and/or condition.

An individual who is “susceptible to” a disease, disorder, and/or condition has not been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

### DETAILED DESCRIPTION

### Method for Preparing the Nanoparticle Suspensions

Among other things, the present invention relates to a formulation of the derivative of 4,9-dihydroxy-naphtho[2,3-b]furan aliphatic acid ester which is as shown in formula I:

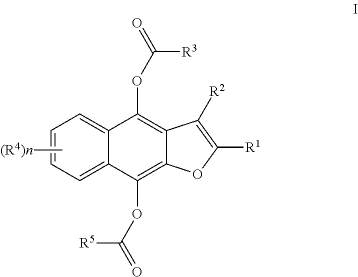

or a pharmaceutically acceptable salt thereof;

wherein:
n is 0-4;
R¹ is independently halogen; —NO₂; —CN; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C₁₋₁₂ aliphatic, 3- to 14-membered carbocyclyl; 3- to 14-membered heterocyclyl; 6- to 14-membered aryl; or 5- to 14-membered heteroaryl; or
R¹ and R² are taken together with their intervening atoms to form an optionally substituted ring selected from 3- to 14-membered carbocycle, 3- to 14-membered heterocycle, 6- to 14-membered aryl, or 5- to 14-membered heteroaryl;
R² is independently hydrogen; halogen; —NO₂; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C_{1-12 aliphatic;} 3- to 14-membered carbocyclyl; 3- to 14-membered heterocyclyl; 6- to 14-membered aryl; or 5- to 14-membered heteroaryl; or
R¹ and R² are taken together with their intervening atoms to form an optionally substituted ring selected from 3- to 14-membered carbocycle, 3- to 14-membered heterocycle, 6- to 14-membered aryl, or 5- to 14-membered heteroaryl;
each R³ and R⁵ is independently an optionally substituted group selected from C₁₋₂₁ aliphatic;
each R⁴ is independently halogen; —NO₂; —CN; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C₁₋₁₂ aliphatic; 3- to 14-membered carbocyclyl; 3- to 14-membered heterocyclyl; 6- to 14-membered aryl; or 5- to 14-membered heteroaryl, or:
two R⁴ groups on adjacent carbon atoms are taken together with their intervening atoms to form an optionally substituted ring selected from 3- to 14-membered carbocycle; 3- to 14-membered heterocycle; a 6- to 14-membered aryl ring; or a 5- to 14-membered heteroaryl ring;
each R is independently hydrogen or an optionally substituted group selected from C₁₋₁₂ aliphatic; 3- to 14-membered carbocyclyl; 3- to 14-membered heterocyclyl; a 6- to 14-membered aryl; or 5- to 14-membered heteroaryl.

Exemplary compounds of formula I are set forth in Table 1 below.

TABLE 1

| | |
|---|---|
| 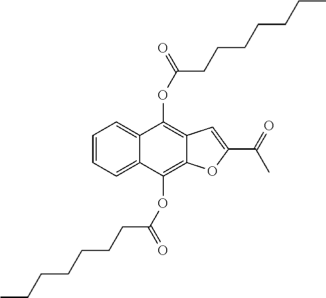 [SMILES: CCCCCCCC(=O)Oc1c2ccccc2c(OC(=O)CCCCCCC)c2oc(C(C)=O)cc12] | Compound I |
| 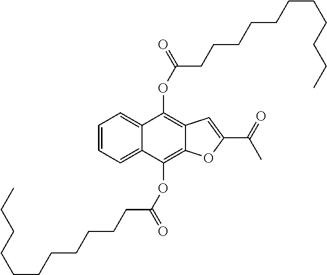 [SMILES: CCCCCCCCCCCC(=O)Oc1c2ccccc2c(OC(=O)CCCCCCCCCCC)c2oc(C(C)=O)cc12] | Compound II |
| 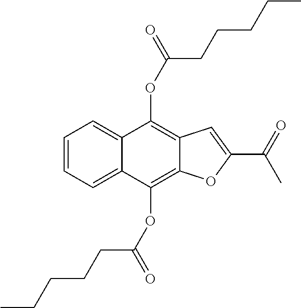 [SMILES: CCCCCC(=O)Oc1c2ccccc2c(OC(=O)CCCCC)c2oc(C(C)=O)cc12] | Compound III |

The present invention provides, in part, a method for preparing an aqueous nanoparticle suspension of a compound of formula I. The method comprises: (1) dissolving a compound of formula I and optionally pharmaceutically acceptable surfactant(s) in a water-miscible organic solvent to form an organic solution; (2) dissolving optionally pharmaceutically acceptable agent(s) and/or optionally pharmaceutically acceptable surfactant(s) in water to form an aqueous solution; and (3) mixing the organic solution and the aqueous solution to form a nanoparticle suspension in which particles have a median particle size (D50) in a range from about 10 nm to about 5000 nm.

In certain embodiments, a compound of formula I is a derivative of 4,9-dihydroxy-naphtho[2,3-b]furan aliphatic acid ester. In other certain embodiments, a compound of formula I is 2-acetyl-4,9-dihydroxy-naphtho[2,3-b]furan aliphatic acid ester. In certain other embodiments, a compound of formula I is 2-acetyl-4,9-dihydroxy-naphtho[2,3-b]furan octanoic acid ester. In certain other embodiments, a compound of formula I is 2-acetyl-4,9-dihydroxy-naphtho[2,3-b]furan dodecanoic acid ester. In certain other embodiments, a compound of formula I is 2-acetyl-4,9-dihydroxy-naphtho[2,3-b]furan hexanoic acid ester.

In certain embodiments, the water-miscible organic solvent includes ethanol, N-methyl-2-pyrrolidinone, 2-pyrrolidone, dimethyl sulfoxide, dimethylacetamide, polyethylene glycol, propylene glycol, and mixtures thereof. In certain other embodiments, the water-miscible organic solvent includes ethanol, N-methyl-2-pyrrolidinone, dimethylacetamide, polyethylene glycol, propylene glycol, and mixtures thereof. In yet other embodiments, the water miscible organic solvent includes dimethylacetamide, polyethylene glycol, and mixtures thereof. In further other embodiments, the water miscible organic solvent includes dimethylacetamide, PEG 300, PEG 400, and mixtures thereof.

In certain embodiments, the optionally pharmaceutically acceptable surfactant in the water-miscible organic solvent includes glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers, mixtures of sucrose stearate and sucrose distearate, random copolymers of vinyl acetate and vinyl pyrrolidone, deoxycholic acid, glycodeoxycholic acid, taurocholic acid, and mixtures thereof. In certain other embodiments, the optionally pharmaceutically acceptable surfactant in the water-miscible organic solvent includes PEG-phospholipids, PEG-cholesterol, PEG-cholesterol derivatives, PEG-vitamin A, PEG-vitamin E, PEG-glycerol mono-(or di-)fatty acid esters, poly(ethylene glycol) mono-fatty acid esters, polyoxyethylene alkyl ethers, polyoxyethylene castor oil derivatives, polyoxyethylene sorbitan fatty acid esters, poloxamers, and mixtures thereof.

In certain embodiments, the water-miscible organic solvent solution contains 0.1-200 mg/ml of a compound of formula I and 0-500 mg/ml of the pharmaceutically acceptable surfactant(s). In certain other embodiments, the water-miscible organic solvent solution contains 0.5-100 mg/ml of a compound of formula I and 0-200 mg/ml of the pharmaceutically acceptable surfactant(s). In yet other embodiments, the water-miscible organic solvent solution contains 3-50 mg/ml of a compound of formula I and 0-100 mg/ml of the pharmaceutically acceptable surfactant(s). In further other embodiments, the water-miscible organic solvent solution contains 5-30 mg/ml of a compound of formula I and 0-50 mg/ml of the pharmaceutically acceptable surfactant(s).

In certain embodiments, the optionally pharmaceutically acceptable agent in the aqueous solution is selected from mannitol, lactose, maltitol, maltodextrin, maltose, dextrates, dextrin, dextrose, fructose, sorbitol, glucose, sucrose, gelatin, alginic acid, and its salt, sodium benzoate, sodium chloride, and mixtures thereof. In certain other embodiments, the optionally pharmaceutically acceptable agent in the aqueous solution is selected from mannitol, maltitol, maltose, dextrose, fructose, sorbitol, glucose, sucrose, sodium benzoate, sodium chloride, and mixtures thereof. In yet other embodiments, the optionally pharmaceutically acceptable agent in the aqueous solution is selected from dextrose, glucose, sodium chloride, and mixtures thereof. In further other embodiments, the optionally pharmaceutically acceptable agent in the aqueous solution is sodium chloride.

In certain embodiments, the optionally pharmaceutically acceptable surfactant in the aqueous solution is selected from glycerol mono-(or di-)fatty acid esters, lecithin, phospholipids (such as phosphatidyl choline, phosphatidyl ethanolamine, phosphatidyl inositol, sphingomyelin, and the like), cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, albumin, poloxamers, mixtures of sucrose stearate and sucrose distearate, random copolymers of vinyl acetate and vinyl pyrrolidone, deoxycholic acid, glycodeoxycholic acid, taurocholic acid, and mixtures thereof. In certain other embodiments, the optionally pharmaceutically acceptable surfactant in the aqueous solution is selected from polyoxyethylene castor oil derivatives, polyoxyethylene sorbitan fatty acid esters, albumin, poloxamers, and mixtures thereof. In yet other embodiments, the optionally pharmaceutically acceptable surfactant in the aqueous solution is selected from albumin and poloxamers.

In certain embodiments, the aqueous solution contains about 0-200 mg/ml of the optionally pharmaceutically acceptable agent(s) and about 0-200 mg/ml of the optionally pharmaceutically acceptable surfactant(s). In certain other embodiments, the aqueous solution contains about 0-100 mg/ml of the optionally pharmaceutically acceptable agent(s) and about 0-100 mg/ml of the optionally pharmaceutically acceptable surfactant(s). In yet other embodiments, the aqueous solution contains about 3-30 mg/ml of the optionally pharmaceutically acceptable agent(s) and about 5-50 mg/ml of the optionally pharmaceutically acceptable surfactant(s).

In certain embodiments, about 1 to 1000 times volume of the aqueous solution is added into the organic solution of a compound of formula I while stirring or vortexing. In certain other embodiments, about 2 to 20 times volume of the aqueous solution is added into the organic solution of a compound of formula I while stirring or vortexing. In yet other embodiments, the organic solution of a compound of formula I is added into about 1 to 1000 times volume of the aqueous solution while stirring or vortexing. In further other embodiments, the organic solution of a compound of formula I is added into about 2 to 20 times volume of the aqueous solution while stirring or vortexing.

In certain embodiments, the nanoparticle suspension prepared according to the method described herein has a median particle size (D50) less than about 5000 nm. In certain other embodiments, the nanoparticle suspension has a median particle size (D50) less than about 2000 nm. In yet other embodiments, the nanoparticle suspension has a median particle size (D50) less than about 500 nm. In further other embodiments, the nanoparticle suspension has a median particle size (D50) less than about 200 nm.

### Compositions of the Nanoparticle Suspensions

The present invention provides, in part, an aqueous nanoparticle suspension comprising:
about 0.1-20 mg/ml of a compound of formula I;
about 0-200 mg/ml of pharmaceutically acceptable agent(s);
about 0-200 mg/ml of pharmaceutically acceptable surfactant(s); and
about 0.1-50% by volume of water-miscible organic solvent or solvent mixture.

Among other things, a compound of formula I is a pharmaceutically active compound which is as shown in formula I:

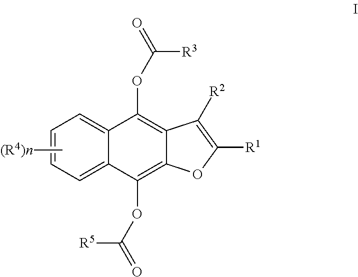

or a pharmaceutically acceptable salt thereof;

wherein each of n, R¹, R², R³, R⁴, and R⁵ is as defined and described herein.

In some embodiments, R¹ is —C(O)R, wherein R is optionally substituted C₁₋₁₂ aliphatic. In some embodiments, each R² and R⁴ is independently hydrogen or halogen. In some embodiments, each R³ and R⁵ is independently C₅₋₁₇ aliphatic.

In some embodiments, R¹ is —C(O)CH₃. In some embodiments, each R² and R⁴ is hydrogen. In some embodiments, each R³ and R⁵ is n-heptyl.

In some embodiments, the pharmaceutically acceptable agent is selected from, but not limited to, mannitol, lactose, maltitol, maltodextrin, maltose, dextrates, dextrin, dextrose, fructose, sorbitol, glucose, sucrose, gelatin, alginic acid, and its salt, sodium benzoate, sodium chloride, and mixtures thereof. The preferred pharmaceutically acceptable agent is selected from mannitol, maltitol, maltose, dextrose, fructose, sorbitol, glucose, sucrose, sodium benzoate, sodium chloride, and mixtures thereof. The more preferred pharmaceutically acceptable agent is selected from mannitol, maltitol, maltose, dextrose, sorbitol, glucose, sodium chloride, and mixtures thereof. The most preferred pharmaceutically acceptable agent is sodium chloride.

In some embodiments, the pharmaceutically acceptable surfactant is selected from glycerol mono-(or di-)fatty acid esters, lecithin, phospholipids (such as phosphatidyl choline, phosphatidyl ethanolamine, phosphatidyl inositol, sphingomyelin, and the like), PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, albumin, poloxamers, mixtures of sucrose stearate and sucrose distearate, random copolymers of vinyl acetate and vinyl pyrrolidone, deoxycholic acid, glycodeoxycholic acid, taurocholic acid, and mixtures thereof. The preferred surfactant is selected from lecithin, phospholipids (such as phosphatidyl choline, phosphatidyl ethanolamine, phosphatidyl inositol, sphingomyelin, and the like), PEG-phospholipids, PEG-cholesterol, PEG-cholesterol derivatives, PEG-vitamin A, PEG-vitamin E, PEG-glycerol mono-(or di-)fatty acid esters, poly(ethylene glycol) mono-fatty acid esters, polyoxyethylene alkyl ethers, polyoxyethylene castor oil derivatives, polyoxyethylene sorbitan fatty acid esters, albumin, poloxamers, and mixtures thereof. The more preferred surfactant is selected from PEG-phospholipids, PEG-cholesterol, PEG-cholesterol derivatives, PEG-vitamin A, PEG-vitamin E, PEG-glycerol mono-(or di-)fatty acid esters, poly(ethylene glycol) mono-fatty acid esters, polyoxyethylene alkyl ethers, polyoxyethylene castor oil derivatives, polyoxyethylene sorbitan fatty acid esters, albumin, poloxamers, and mixtures thereof. The most preferred surfactant is poloxamer 188.

Among other things, the preferred water-miscible organic solvent is selected from ethanol, N-methyl-2-pyrrolidinone, 2-pyrrolidone, dimethyl sulfoxide, dimethylacetamide, polyethylene glycol, propylene glycol, and mixtures thereof. The more preferred water-miscible organic solvent is selected from ethanol, N-methyl-2-pyrrolidinone, dimethylacetamide, polyethylene glycol, propylene glycol, and mixtures thereof. The most preferred water-miscible organic solvent is a mixture of dimethylacetamide and polyethylene glycol 300.

In certain embodiments, the inventive aqueous nanoparticle suspension comprises: about 0.1-20 mg/ml of a compound of formula I; about 0-200 mg/ml of pharmaceutically acceptable agent(s); about 0-200 mg/ml of pharmaceutically acceptable surfactant(s); and about 0.1-50% by volume of water-miscible organic solvent or solvent mixture. In certain other embodiments, the inventive aqueous nanoparticle suspension comprises: about 0.2-10 mg/ml of a compound of formula I; about 1-100 mg/ml of pharmaceutically acceptable agent(s); about 0-100 mg/ml of pharmaceutically acceptable surfactant(s); and about 0.5-40% by volume of water-miscible organic solvent or solvent mixture. In yet other embodiments, the inventive aqueous nanoparticle suspension comprises: about 0.3-8 mg/ml of a compound of formula I; about 1-80 mg/ml of pharmaceutically acceptable agent(s); about 1-80 mg/ml of pharmaceutically acceptable surfactant(s); and about 1-30% by volume of water-miscible organic solvent or solvent mixture.

In certain embodiments, the nanoparticle suspension prepared according to the method described herein has a median particle size (D50) less than about 5000 nm. In certain other embodiments, the nanoparticle suspension has a median particle size (D50) less than about 2000 nm. In yet other embodiments, the nanoparticle suspension has a median particle size (D50) less than about 500 nm. In further other embodiments, the nanoparticle suspension has a median particle size (D50) less than about 200 nm.

The present invention further provides a kit which can be used by those of ordinary skill in this art for preparing an aqueous nanoparticle suspension of a compound of formula I. In certain embodiments, the kit comprises: a water-miscible organic solvent solution containing about 0.1-200 mg/ml of a compound of formula I and 0-500 mg/ml of pharmaceutically acceptable surfactant(s) or ingredients for preparing such organic solution; an aqueous solution containing about 0-200 mg/ml of pharmaceutically acceptable agent(s) and about 0-200 mg/ml of pharmaceutically acceptable surfactant(s) or ingredients for preparing such aqueous solution; and instruction on how to mix the organic solution and the aqueous solution to form a nanoparticle suspension and also on how to prepare the organic solution and/or the aqueous solution if components instead of finished solution(s) are provided in the kit. In certain other embodiments, the kit comprises: a water-miscible organic solvent solution containing about 0.5-100 mg/ml of a compound of formula I and 0-100 mg/ml of pharmaceutically acceptable surfactant(s) or ingredients for preparing such organic solution; an aqueous solution containing about 5-100 mg/ml of pharmaceutically acceptable agent(s) and about 5-100 mg/ml of pharmaceutically acceptable surfactant(s) or ingredients for preparing such aqueous solution; and instruction on how to mix the organic solution and the aqueous solution to form a nanoparticle suspension and also on how to prepare the organic solution and/or the aqueous solution if components instead of finished solution(s) are provided in the kit.

### Uses of the Nanoparticle Suspensions

The present invention provides nanoparticle suspensions prepared according to the method as described herein. The nanoparticle suspensions may be used in vitro or in vivo. In some embodiments, the inventive nanoparticle suspensions are used in vitro for research or clinical purposes (e.g., determining the susceptibility of a patient's disease to a compound of formula I, researching the mechanism of action, elucidating a cellular pathway or process). In some embodiments, the inventive nanoparticle suspensions are used in vivo as medicine.

The nanoparticle suspensions can be used for parenteral administration as medicine, or for enteral and topical administration as medicine, or for oral administration as medicine. In some embodiments, the nanoparticle suspensions are used for parenteral administration. In some embodiments, the nanoparticle suspensions are used for enteral administration. In some embodiments, the nanoparticle suspensions are used for topical administration. In some embodiments, the nanoparticle suspensions are used for oral administration.

In some embodiments, the present invention provides methods of treating a subject suffering from or susceptible to a disease, disorder, or condition, the method comprising administering to the subject a therapeutically effective amount of a compound of formula I in a nanoparticle suspension prepared according to the method as described herein.

In some embodiments, the present invention provides a method of treating a subject suffering from or susceptible to a proliferative disease, disorder, or condition, the method comprising administering to the subject a therapeutically effective amount of a compound of formula I in a nanoparticle suspension prepared according to the method as described herein. In certain embodiments, the proliferative disease is a benign neoplasm. In certain embodiments, the proliferative disease is cancer. In certain embodiments, the proliferative disease is an inflammatory disease. In certain embodiments, the proliferative disease is an autoimmune disease. In certain embodiments, the proliferative disease is diabetic retinopathy.

The nanoparticle suspension of a compound of formula I may be used in the treatment of neoplasms. In certain embodiments, the neoplasm is a benign neoplasm. In other embodiments, the neoplasm is a malignant neoplasm.

In some embodiments, the present invention provides a method of treating a subject suffering from or susceptible to cancer, the method comprising administering to the subject a therapeutically effective amount of a compound of formula I in a nanoparticle suspension prepared according to the method as described herein. In some embodiments, the cancer is a hematological malignancy. In certain embodiments, the cancer is a solid tumor. Exemplary cancers that may be treated using the nanoparticle suspension containing a compound of formula I include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, breast cancer, gastric cancer, eye cancer, nasopharyngeal cancer, gallbladder cancer, laryngeal cancer, oral cancer, penile cancer, glandular tumors, rectal cancer, small intestine cancer, head and neck cancer, multiple myeloma, colorectal carcinoma, kaposi sarcoma, ewing's sarcoma, osteosarcoma, leiomyosarcoma, glioma, meningioma, medulloblastoma, melanoma, urethral cancer, and vaginal cancer, to name but a few.

Hematological malignancies are types of cancers that affect the blood, bone marrow, and/or lymph nodes. Examples of hematological malignancies that may be treated using the nanoparticle suspension containing a compounds of formula I include, but are not limited to, acute lymphoblastic leukemia (ALL), acute myelogenous leukemia (AML), chronic myelogenous leukemia (CML), chronic lymphocytic leukemia (CLL), hairy cell leukemia, Hodgkin's lymphoma, non-Hodgkin's lymphoma, cutaneous T-cell lymphoma (CTCL), peripheral T-cell lymphoma (PTCL), Mantle cell lymphoma, B-cell lymphoma, acute lymphoblastic T cell leukemia (T-ALL), acute promyelocytic leukemia, and multiple myeloma.

The nanoparticle suspensions containing compounds of formula I may also be used to treat a refractory or relapsed malignancy. In certain embodiments, the cancer is a refractory and/or relapsed hematological malignancy. For example, the cancer may be resistant to a particular chemotherapeutic agent.

In some embodiments, the present invention provides a method of inhibiting or reducing cancer stem cell survival and/or self renewal with an effective amount of a compound of formula I in a nanoparticle suspension prepared according to the method as described herein.

The nanoparticle suspensions containing compounds of formula I may also be used to treat and/or kill cells in vitro or in vivo. In certain embodiments, a cytotoxic concentration of a compound of formula I in the nanoparticle suspension is contacted with the cells in order to kill them. In some embodiments, a sublethal concentration of a compound of formula I in the nanoparticle suspension is used to treat the cells. In certain embodiments, the concentration of a compound of formula I ranges from 0.1 nM to 100 μM. In certain embodiments, the concentration of a compound of formula I ranges from 0.01 μM to 100 μM. In certain embodiments, the concentration of a compound of formula I ranges from 0.1 μM to 50 μM. In certain embodiments, the concentration of a compound of formula I ranges from 1 μM to 10 μM. In certain embodiments, the concentration of a compound of formula I ranges from 1 μM to 10 μM, more particularly 1 μM to 5 μM.

Any type of cell may be tested or killed with a compound of formula I in the nanoparticle suspension. Such cells may be derived from any animal, plant, bacterial, or fungal source, and may be at any stage of differentiation or development. In certain embodiments, cells are animal cells. In certain embodiments, cells are vertebrate cells. In certain embodiments, cells are mammalian cells. In certain embodiments, cells are human cells. Cells may be derived from a male or female human in any stage of development. In certain embodiments, cells are primate cells. In other embodiments, cells are derived from a rodent (e.g., mouse, rat, guinea pig, hamster, gerbil). In certain embodiments, cells are derived from a domesticated animal such as a dog, cat, cow, goat, pig, etc. Cells may also be derived from a genetically engineered animal or plant, such as a transgenic mouse.

Cells used in accordance with the present invention may be wild type or mutant cells, and may be genetically engineered. In certain embodiments, cells are normal cells. In certain embodiments, cells are hematological cells. In certain embodiments, cells are white blood cells. In certain particular embodiments, cells are precursors of white blood cells (e.g., stem cells, progenitor cells, blast cells). In certain embodiments, cells are neoplastic cells. In certain embodiments, cells are cancer cells. In certain embodiments, cells are derived from a hematological malignancy. In other embodiments, cells are derived from a solid tumor. For example, cells may be derived from a patient's tumor (e.g., from a biopsy or surgical excision). In certain embodiments, cells are derived from a blood sample from the subject or from a bone marrow biopsy. In certain embodiments, cells are derived from a lymph node biopsy. Such testing for cytotoxicity may be useful in determining whether a patient will respond to a particular combination therapy. Such testing may also be useful in determining the dosage needed to treat the malignancy. This testing of the susceptibility of a patient's cancer to a compound of formula I in the nanoparticle suspension would prevent the unnecessary administration of drugs with no effect to the patient. The testing may also allow the use of a lower dose of a compound of formula I if the patient's cancer is particularly susceptible to the compound of formula I.

In certain embodiments, cells are derived from cancer cells lines. For example, in certain embodiments, cells are hematopoietic progenitor cells such as CD34⁺ bone marrow cells. In certain embodiments, cells are A549, DLD1, SW480, LOVO, HT-29, U-20S, MES-SA, SK-MEL-28, Panc-1, DU-145, CNE, U251, Eca-109, MGC80-3, SGC-7901, QGY-7701, BEL-7404, PLC/PRF/5, Huh-7, MOLT-3 (acute lymphoblastic T-cell), SKNLP (neuroblastoma), PC9 (adenocarcinoma), H1650 (adenocarcinoma), H1975 (adenocarcinoma), H2030 (adenocarcinoma), H3255 (adenocarcinoma), TC71 (Ewing's sarcoma), HTP-15 (glioblastoma), A431 (epithelial carcinoma), HeLa (cervical adenocarcinoma), or WD0082 (well-differentiated liposarcoma) cells. In certain embodiments, cell lines are resistant to a particular chemotherapeutic agent.

In some embodiments, the present invention provides a method of treating a subject suffering from or susceptible to obesity or an obesity-related disorder or condition, the method comprising administering to the subject a therapeutically effective amount of a compound of formula I in the nanoparticle suspension.

In some embodiments, the present invention provides a method of treating a subject suffering from or susceptible to diabetes, the method comprising administering to the subject a therapeutically effective amount of a compound of formula I in the nanoparticle suspension.

In some embodiments, the present invention provides a method of treating a subject suffering from or susceptible to a metabolic disease, disorder, or condition, the method comprising administering to the subject a therapeutically effective amount of a compound of formula I in the nanoparticle suspension.

In some embodiments, the present invention provides a method of treating a subject suffering from or susceptible to a degenerative disease, disorder, or condition, the method comprising administering to the subject a therapeutically effective amount of a compound of formula I in the nanoparticle suspension.

In some embodiments, the present invention provides a method of treating a subject suffering from or susceptible to a disease, disorder, or condition associated with mitochondrial dysfunction, the method comprising administering to the subject a therapeutically effective amount of a compound of formula I in the nanoparticle suspension.

In some embodiments, the present invention provides a method of treating a subject suffering from or susceptible to a cardiovascular disease, disorder, or condition, the method comprising administering to the subject a therapeutically effective amount of a compound of formula I in the nanoparticle suspension. In some embodiments, the disease, disorder, or condition is selected from the group consisting of hypertension, congestive heart failure, heart attack, hypertensive heart disease, atherosclerosis, coronary artery disease, angina, ischemia, ischemic stroke.

In some embodiments, the nanoparticle suspension containing compound of formula I may be useful to treat other diseases, disorders, or conditions as described in WO 2009/036059 and WO 2006/088315, the entire contents of each of which are hereby incorporated by reference.

In certain embodiments, the nanoparticle suspensions of the present invention can be employed in combination therapies, that is, the nanoparticle suspensions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved.

In certain embodiments, other therapies or anticancer agents that may be used in combination with the nanoparticle suspension of the present invention include surgery, radiotherapy (γ-radiation, neutron beam radiotherapy, electron beam radiotherapy, proton therapy, brachy therapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabine, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies see The Merck Manual, Eighteenth Ed. 2006, the entire contents of which are hereby incorporated by reference.

In certain embodiments, inventive nanoparticle suspensions are useful in treating a subject in clinical remission. In some embodiments, the subject has been treated by surgery and may have limited unresected disease.

If desired, the effective daily dose of a compound of formula I in the inventive nanoparticle suspension may be administered as two, three, four, five, six, or more sub-doses administered separately at appropriate intervals throughout the day, optionally, in unit dosage forms.

Actual dosage levels of a compound of formula I in the inventive nanoparticle suspension may be varied by, so as to obtain an amount of a compound of formula I that is effective to achieve the desired therapeutic response for a particular patient, composition and mode of administration, without being toxic to the patient.

The selected dosage level will depend upon a variety of factors including the activity of the particular compound of formula I in the nanoparticle suspension of the present invention employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compound of formula I in the nanoparticle suspension at levels lower than that required to achieve the desired therapeutic effect and then gradually increase the dosage until the desired effect is achieved.

In some embodiments, the nanoparticle suspension containing a compound of formula I is provided to a subject chronically. Chronic treatments include any form of repeated administration for an extended period of time, such as repeated administrations for one or more months, between a month and a year, one or more years, or longer. In some embodiments, a chronic treatment involves administering the nanoparticle suspension containing a compound of formula I repeatedly over the life of the subject. In some embodiments, chronic treatments involve regular administrations, for example one or more times a day, one or more times a week, or one or more times a month. In general, a suitable dose such as a daily dose of a compound of formula I in the nanoparticle suspension will be that amount of the compound that is the lowest dose effective to produce a therapeutic effect. Such an effective dose will generally depend upon the factors described above. Generally doses of the compounds in the nanoparticle suspensions for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

In certain embodiments, a therapeutically effective amount of a compound of formula I in the nanoparticle suspension is from about 1 mg/m² to about 5,000 mg/m² (I.V.) or from about 1 mg/m² to about 50,000 mg/m² (PO). In certain embodiments, a therapeutically effective amount of a compound of formula I in the nanoparticle suspension is from about 2 mg/m² to about 3,000 mg/m² (I.V.) or from about 10 mg/m² to about 30,000 mg/m² (PO).

In certain embodiments, a compound of formula I is administered in a suitable dosage form prepared by combining a therapeutically effective amount of a compound of formula I in the nanoparticle suspension with at least one excipient or carrier or diluent according to conventional procedures well known in the art. The dosage form for treatment of cancer may be injected directly into tumors, injected into the blood stream or body cavities or taken orally or applied through the skin with patches.

### EXEMPLIFICATION

### Example 1

### Preparation of 2-acetyl-naphtho[2,3-b]furan-4,9-dione 1. Preparation of 3-buten-2-one

To a 1 L round-bottom flask, 600 ml of 4-hydroxy-2-butanone, 100 ml of water, 50 ml of methanol and 20 ml of 85% phosphoric acid were added. The mixture was stirred at room temperature for 30 minutes, and then distilled under reduced pressure (100-300 mmHg), where a fraction at the boiling point of 65-80° C. was collected. To the collected fraction, 80 grams of sodium chloride was added. The resulting mixture was stirred at 4° C. for 1 hour, and then the top organic layer was separated with a funnel, dried with anhydrous sodium sulfate, and stored at 4° C.

### 2. Preparation of 2-acetyl-naphtho[2,3-b]dihydrofuran-4,9-dione

To a 500 ml round-bottom flask containing 16.1 grams (0.23 mol) of 3-buten-2-one and 40 ml of dichloromethane cooled in an ice-salt bath, 36.7 grams (0.23 mol) of bromine diluted in 10 ml of dichloromethane was added dropwise in 15 minutes. The mixture was washed with 50 ml of water, dried with anhydrous sodium sulfate, and evaporated to remove dichloromethane. 43.4 grams (0.19 mol) of the residue was transferred into an 1 L round-bottom flask, diluted with 40 ml of DMF and cooled in an ice-salt bath. While stirring vigorously, 27.3 grams (0.18 mol) of 1,8-Diazabicyclo[5.4.0]undec-7-ene (DBU) in 50 ml of DMF was added dropwise in 15 minutes. To the mixture, 31.4 grams (0.18 mol) of 2-hydroxy-1,4-naphthoquinone was added, and the ice-salt bath was removed. While stirring vigorously and open in air, 25.8 grams (0.17 mol) of DBU in 50 ml of DMF was added dropwise in 30 minutes at room temperature. After stirring for 4 hours, 500 ml of ice cooled water was added into the mixture. The crude product was filtered, washed with water, 5% aqueous sodium bicarbonate, water, 2% aqueous acetic acid solution, and ice-cooled ethanol, successively. Pure product (21.8 grams, yield 50.1%) was obtained by crystallization in ethyl acetate, and characterized by ¹H NMR and mass spectrum. ¹H NMR (in DMSO) δ 2.61 (s, 3H), 7.91-7.95 (m, 2H), 8.06 (s, 1H), 8.13-8.17 (m, 2H). Mass (M+H) is 241.

### Example 2

### Preparation of 2-acetyl-4,9-bis(octanoyloxy)-naphtho[2,3-b]furan (Compound I)

To a 1 L round-bottom flask was added 30 grams (125 mmoles) of 2-acetyl-naphtho[2,3-b]furan-4,9-dione (prepared as described in example 1), 300 ml of dimethylformide, 87.1 ml of triethylamine (625 mmol), 30 grams (470 mmol) of zinc powder, 3 grams of tetrabutylammonium bromide, and 109 grams (625 mmol) of sodium hydrosulfite. The mixture was isolated from air by nitrogen atmosphere or by sealing from air, and stirred vigorously at room temperature for 20 minutes. Then 81.3 grams (500 mmoles) of caprylic chloride was added dropwise with syringe in 30 minutes, and the resulting mixture was stirred vigorously at room temperature for additional 3 hours. The reaction mixture was filtered, and the solid was washed with 50 ml of dimethylformide. To the combined filtrate, 1000 ml of 5% acetic acid aqueous solution was added, and the resulting mixture was stirred for 1 hour. The crude solid product was collected by filtration, washed with 200 ml of water twice, crystallized in ethanol and re-crystallized in acetone/water (8:1). 15.0 grams (30.4 mmoles, yield 24.3%) of product was obtained and characterized by ¹H NMR. ¹H NMR (in CDCl₃) δ 0.93-0.97 (m, 6H), 1.37-1.39 (m, 9H), 1.42-1.47 (m, 4H), 1.51-1.59 (m, 3H), 1.90-2.00 (m, 4H), 2.65 (s, 3H), 2.83-2.91 (m, 4H), 7.46 (s, 1H), 7.51-7.61 (m, 2H), 7.99-8.02 (m, 2H).

### Example 3

### Preparation of 2-acetyl-4,9-bis(dodecanoyloxy)-naphtho[2,3-b]furan (Compound II)

To a 250 ml round-bottom flask was added 5 grams (20.8 mmoles) of 2-acetyl-naphtho[2,3-b]furan-4,9-dione (prepared as described in example 1), 100 ml of dimethylformide, 11.6 ml of triethylamine (83.3 mmol), 5 grams (78.1 mmol) of zinc powder, 1 gram of tetrabutylammonium bromide, and 18.1 grams (104.2 mmol) of sodium hydrosulfite. The mixture was isolated from air by nitrogen atmosphere or by sealing from air, and stirred vigorously at room temperature for 20 minutes. Then, 13.7 ml (62.5 mmoles) of lauroyl chloride was added dropwise with syringe in 30 minutes, and the resulting mixture was stirred vigorously at room temperature for additional 5 hours. The reaction mixture was filtered, and the solid was washed with 150 ml of ethyl acetate three times. The combined filtrate was transferred into a separatory funnel, washed with 150 ml of 3% citric acid aqueous solution twice, and the resulting aqueous phases were combined and reversely extracted with 100 ml of ethyl acetate. The combined organic phase was washed with 200 ml of water four times, and then dried with anhydrous sodium sulfate. The organic solution was filtered and evaporated to dryness under reduced pressure. The residue was crystallized in ethanol. 4.1 grams (6.76 mmoles, yield 32.5%) of product was obtained and characterized by ¹H NMR. ¹H NMR (in CDCl₃) δ 0.89-0.93 (m, 6H), 1.28-1.60 (m, 32H), 1.90-2.00 (m, 4H), 2.65 (s, 3H), 2.83-2.91 (m, 4H), 7.46 (s, 1H), 7.51-7.61 (m, 2H), 7.99-8.02 (m, 2H).

### Example 4

### Preparation of 2-acetyl-4,9-bis(hexanoyloxy)-naphtho[2,3-b]furan (Compound III)

To a 250 ml round-bottom flask was added 5 grams (20.8 mmoles) of 2-acetyl-naphtho[2,3-b]furan-4,9-dione (prepared as described in example 1), 100 ml of dimethylformide, 14.6 ml of triethylamine (104.2 mmol), 5 grams (78.1 mmol) of zinc powder, 1 gram of tetrabutylammonium bromide, and 18.1 grams (104.2 mmol) of sodium hydrosulfite. The mixture was isolated from air by nitrogen atmosphere or by sealing from air, and stirred vigorously at room temperature for 20 minutes. Then, 19.2 grams (83.3 mmoles) of caproic anhydride was added dropwise with syringe in 30 minutes, and the resulting mixture was stirred vigorously at room temperature for additional 5 hours. The reaction mixture was filtered, and the solid was washed with 150 ml of ethyl acetate three times. The combined filtrate was transferred into a separatory funnel, washed with 150 ml of 3% citric acid aqueous solution twice, and the resulting aqueous phases were combined and reversely extracted with 100 ml of ethyl acetate. The combined organic phases was washed with 200 ml of water four times, and then dried with anhydrous sodium sulfate. The organic solution was filtered and evaporated to dryness under reduced pressure. The residue was crystallized in ethanol. 4.2 grams (9.59 mmoles, yield 46.1%) of product was obtained and characterized by ¹H NMR and mass spectrum. ¹H NMR (in CDCl₃) δ 0.89-0.93 (t, J=7, 6H), 1.45-1.61 (m, 8H), 1.91-2.01 (m, 4H), 2.65 (s, 3H), 2.83-2.91 (m, 4H), 7.46 (s, 1H), 7.51-7.61 (m, 2H), 7.99-8.02 (m, 2H). Mass (M+H) is 439.

### Example 5

### Preparation of Nanoparticle Suspension (NS-I)

To a 20 ml vial was added 240 mg of 2-acetyl-4,9-bis(octanoyloxy)-naphtho[2,3-b]furan (compound I) and 10 ml of PEG 400. The mixture was heated to 80° C. and stirred vigorously until the mixture became a clear solution. The clear solution was divided into two portions which were diluted with 5 and 11 times volume of 3% (w/v) poloxamer 188 in saline, respectively. The resulting mixtures were 2 mg/ml compound I nanoparticle suspension (NS-I-2) and 4 mg/ml compound I nanoparticle suspension (NS-I-4), in which concentrations of compound I was confirmed by HPLC analysis. At different time points after preparation, a portion of either NS-I-2 or NS-I-4 was diluted 30 to 100 times with water, and then analyzed with Winner 801 (purchased from Jinan Winner Particle Instruments Stock Co., Ltd, Jinan, Shandong, China) for its particle distribution. Particle distribution data are shown below.

| | | Nanoparticle Suspension | | | Nanoparticle Suspension | | |
|---|---|---|---|---|---|---|---|
| | | NS-I-2 | | | NS-I-4 | | |
| Time after | | D10 | D50 | D90 | D10 | D50 | D90 |
| Preparation | | (nm) | (nm) | (nm) | (nm) | (nm) | (nm) |
| 0 | min | 100.4 | 168.3 | 281.5 | 81.0 | 136.8 | 231.0 |
| 30 | min | 77.8 | 147.2 | 279.1 | 75.5 | 132.5 | 232.4 |
| 1 | h | 89.4 | 153.7 | 263.8 | 97.5 | 150.0 | 230.6 |
| 2 | h | 85.1 | 154.1 | 279.1 | 75.6 | 131.0 | 226.5 |
| 3 | h | 103.3 | 170.2 | 280.3 | 91.4 | 146.8 | 235.1 |
| 4 | h | 101.0 | 166.5 | 274.9 | 82.8 | 140.9 | 239.2 |

### Example 6

### Preparation of Nanoparticle Suspension (NS-II)

To a 20 ml vial was added 240 mg of 2-acetyl-4,9-bis(octanoyloxy)-naphtho[2,3-b]furan (compound I), 24 mg of citric acid and 10 ml of PEG 400. The mixture was heated to 80° C. and stirred vigorously until the mixture became a clear solution. The clear solution was diluted with 5 times volume of 3% (w/v) poloxamer 188 in phosphate buffered saline (pH 7.4). The resulting mixture was a 4 mg/ml compound I nanoparticle suspension (NS-II-4), in which concentration of compound I was confirmed by HPLC analysis. At different time points after preparation, a portion of NS-II-4 was diluted 30 to 100 times with water, and then analyzed with Winner 801 (purchased from Jinan Winner Particle Instruments Stock Co., Ltd, Jinan, Shandong, China) for its particle distribution. Particle distribution data are shown below.

| | |
|---|---|
| Time after | Nanoparticle Suspension NS-II-4 |

| | | | |
|---|---|---|---|
| Preparation | D10 (nm) | D50 (nm) | D90 (nm) |

| | | | | |
|---|---|---|---|---|
| 0 | min | 44.5 | 93.3 | 195.5 |
| 30 | min | 43.5 | 90.4 | 187.9 |
| 1 | h | 51.2 | 95.8 | 178.5 |
| 2 | h | 44.2 | 86.5 | 169.7 |

### Example 7

### Preparation of Nanoparticle Suspension (NS-III)

To a 20 ml vial was added 50 mg of 2-acetyl-4,9-bis(dodecanoyloxy)-naphtho[2,3-b]furan (compound II) and 10 ml of PEG 400. The mixture was heated to 80° C. and stirred vigorously until the mixture became a clear solution. The clear solution was diluted with 4 times volume of 6% (w/v) poloxamer 188 in saline. The resulting mixture was a 1 mg/ml compound II nanoparticle suspension (NS-III), in which concentration of compound II was confirmed by HPLC analysis. At different time points after preparation, a portion of NS-III was diluted 10 to 20 times with water, and then analyzed with Winner 801 (purchased from Jinan Winner Particle Instruments Stock Co., Ltd, Jinan, Shandong, China) for its particle distribution. Particle distribution data are shown below.

| Time after | Nanoparticle Suspension NS-III | | |
|---|---|---|---|
| Preparation | D10 (nm) | D50 (nm) | D90 (nm) |
| 0 min | 84.3 | 148.6 | 262.2 |
| 30 min | 81.5 | 160.5 | 315.9 |

### Example 8

### Preparation of Nanoparticle Suspension (NS-IV)

To a 20 ml vial was added 50 mg of 2-acetyl-4,9-bis(hexanoyloxy)-naphtho[2,3-b]furan (compound III) and 10 ml of PEG 400. The mixture was heated to 80° C. and stirred vigorously until the mixture became a clear solution. The clear solution was diluted with 4 times volume of 6% (w/v) poloxamer 188 in saline. The resulting mixture was a 1 mg/ml compound III nanoparticle suspension (NS-IV), in which concentration of compound III was confirmed by HPLC analysis. At different time points after preparation, a portion of NS-IV was diluted 10 to 20 times with water, and then analyzed with Winner 801 (purchased from Jinan Winner Particle Instruments Stock Co., Ltd, Jinan, Shandong, China) for its particle distribution. Particle distribution data are shown below.

| | |
|---|---|
| Time after | Nanoparticle Suspension NS-IV |

| | | | |
|---|---|---|---|
| Preparation | D10 (nm) | D50 (nm) | D90 (nm) |

| | | | | |
|---|---|---|---|---|
| 0 | min | 102.4 | 161.1 | 254.1 |
| 30 | min | 138.4 | 194.5 | 274.0 |
| 1 | h | 146.2 | 211.5 | 306.2 |

### Example 9

### Preparation of Nanoparticle Suspension (NS-V)

To a 20 ml vial was added 40 mg of 2-acetyl-4,9-bis(octanoyloxy)-naphtho[2,3-b]furan (compound I) and 2100 mg of PEG 4000. The mixture was heated in an 80° C. oven with occasional vortex until the mixture became a clear solution. The clear solution was then diluted with 4 times volume of saline. The resulting mixture was a 4 mg/ml compound I nanoparticle suspension (NS-V), in which concentration of compound I was confirmed by HPLC analysis. At different time points after preparation, a portion of NS-V was diluted 30 to 60 times with water, and then analyzed with Winner 801 (purchased from Jinan Winner Particle Instruments Stock Co., Ltd, Jinan, Shandong, China) for its particle distribution. Particle distribution data are shown below.

| Time after | Nanoparticle Suspension NS-V | | |
|---|---|---|---|
| Preparation | D10 (nm) | D50 (nm) | D90 (nm) |
| 0 min | 73.4 | 125.0 | 212.7 |
| 30 min | 86.2 | 139.1 | 224.9 |

### Example 10

### Preparation of Nanoparticle Suspension (NS-VI)

To a 20 ml vial was added 40 mg of 2-acetyl-4,9-bis(octanoyloxy)-naphtho[2,3-b]furan (compound I) and 2100 mg of PEG 20000. The mixture was heated in an 80° C. oven with occasional vortex until the mixture became a clear solution. The clear solution was then diluted with 4 times volume of saline. The resulting mixture was a 4 mg/ml compound I nanoparticle suspension (NS-VI), in which concentration of compound I was confirmed by HPLC analysis. At different time points after preparation, a portion of NS-VI was diluted 30 to 60 times with water, and then analyzed with Winner 801 (purchased from Jinan Winner Particle Instruments Stock Co., Ltd, Jinan, Shandong, China) for its particle distribution. Particle distribution data are shown below.

| Time after | Nanoparticle Suspension NS-VI | | |
|---|---|---|---|
| Preparation | D10 (nm) | D50 (nm) | D90 (nm) |
| 0 min | 64.5 | 127.7 | 252.8 |
| 30 min | 155.8 | 199.5 | 254.6 |

### Example 11

### Preparation of Nanoparticle Suspension (NS-VII)

In a 4 ml vial, 100 mg of 2-acetyl-4,9-bis(octanoyloxy)-naphtho[2,3-b]furan (compound I) was dissolved in 0.5 ml of dimethylacetamide (DMA) to become a 167 mg/ml compound I solution in DMA. 0.272 ml of this solution was then added into molten 2100 mg of PEG 4000 (80° C.) in a 20 ml vial. The mixture was heated in an 80° C. oven with occasional vortex until the mixture became a uniform solution (20 mg/ml compound I solution in DMA/PEG 4000, 1/9, V/V). The clear solution was then diluted with 4 times volume (9.09 ml) of saline. The resulting mixture was a 4 mg/ml compound I nanoparticle suspension (NS-VII), in which concentration of compound I was confirmed by HPLC analysis. At different time points after preparation, a portion of NS-VII was diluted 30 to 60 times with water, and then analyzed with Winner 801 (purchased from Jinan Winner Particle Instruments Stock Co., Ltd, Jinan, Shandong, China) for its particle distribution. Particle distribution data are shown below.

| Time after | Nanoparticle Suspension NS-VII | | |
|---|---|---|---|
| Preparation | D10 (nm) | D50 (nm) | D90 (nm) |
| 0 min | 70.9 | 118.6 | 198.2 |
| 30 min | 74.1 | 124.6 | 209.5 |

### Example 12

### Preparation of Nanoparticle Suspension (NS-VIII)

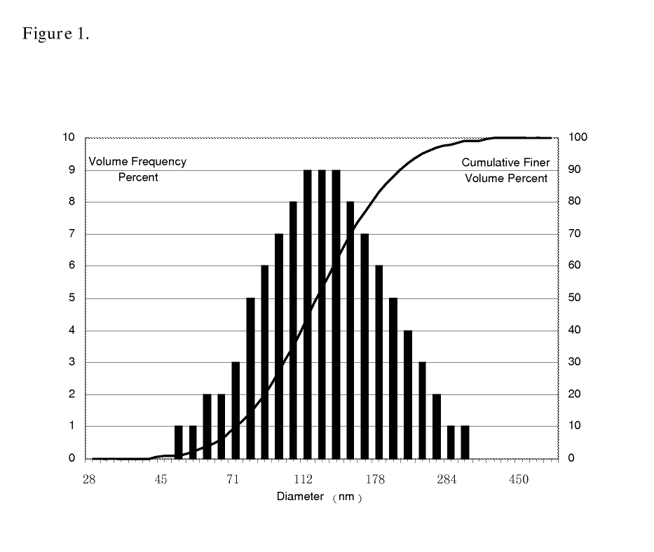

In a 4 ml vial, 160 mg of 2-acetyl-4,9-bis(octanoyloxy)-naphtho[2,3-b]furan (compound I) was dissolved in 1.0 ml of dimethylacetamide (DMA) to become a 141 mg/ml compound I solution in DMA. To a 20 ml vial was added 0.567 ml of 141 mg/ml compound I solution in DMA and 3.433 ml of PEG300 to become a 20 mg/ml compound I solution in DMA/PEG300 (6.87:1). To the solution with continuous vortex, 8 ml of 2% poloxamer 188 in saline was then added quickly (less than 2 seconds). The resulting mixture was a 6.67 mg/ml compound I nanoparticle suspension (NS-VIII-6.67) in an aqueous solution which contained 4.17% DMA, 28.65% PEG300 and 1.33% poloxamer 188. Compound I nanoparticle suspension (NS-VIII-6.67) was further diluted with saline to become a 4.00 mg/ml compound I nanoparticle suspension (NS-VIII-4.00) in an aqueous solution which contained 2.50% DMA, 17.18% PEG300 and 0.80% poloxamer 188. Concentrations of compound I in both NS-VIII-6.67 and NS-VIII-4.00 were confirmed by HPLC analysis. At different time points after preparation, portions of both NS-VIII-6.67 and NS-VIII-4.00 were diluted 30 to 60 times with water, and then analyzed with Winner 801 (purchased from Jinan Winner Particle Instruments Stock Co., Ltd, Jinan, Shandong, China) for their particle distributions (see FIG. 1 for particle distribution of NS-VIII-4.00). Particle distribution data are shown below.

| | | Nanoparticle Suspension | | | Nanoparticle Suspension | | |
|---|---|---|---|---|---|---|---|
| | | NS-VIII-6.67 | | | NS-VIII-4.00 | | |
| Time after | | D10 | D50 | D90 | D10 | D50 | D90 |
| Preparation | | (nm) | (nm) | (nm) | (nm) | (nm) | (nm) |
| 0 | min | 80.98 | 124.90 | 192.44 | 65.63 | 110.19 | 185.29 |
| 2 | h | 92.68 | 138.64 | 207.85 | 65.82 | 112.34 | 192.02 |
| 4 | h | 68.01 | 130.31 | 249.68 | 70.95 | 119.97 | 203.32 |

### Example 13

### Pharmacokinetic Study of Nanoparticle Suspension NS-I-4 in ICR Mice

Nanoparticle suspension NS-I-4, prepared according to the method as described in example 5, was administered intravenously into ICR mice at a dosage of 30 mg/kg. At different time points (5 min, 10 min, 30 min, 1 h, 2 h, 3 h, 4 h, 6 h) after the drug injection, blood samples were withdrawn from the dosed mice, and then were centrifuged at 6,000 rpm for 10 minutes at 4° C. The supernatant plasma samples were fetched and treated with 9 times volume of acetonitrile containing 0.5% trifluoromethane sulfonic acid. The treated plasmas were centrifuged at 12,000 rpm for 15 minutes at 4° C., and then the supernatants were fetched and analyzed with HPLC.

When administered intravenously into ICR mice, compound I is degraded by esterases into active drug 2-acetyl-4,9-dihydroxy-naphtho[2,3-b]furan which can be further metabolized into active metabolite 2-(1-hydroxyl)ethyl-4,9-dihydroxy-naphtho[2,3-b]furan (see scheme 1). During treatment of blood samples before HPLC analysis, active drug 2-acetyl-4,9-dihydroxy-naphtho[2,3-b]furan and active metabolite 2-(1-hydroxyl)ethyl-4,9-dihydroxy-naphtho[2,3-b]furan are both oxidized into 2-acetyl-naphtho[2,3-b]furan-4,9-dione and 2-(1-hydroxyl)ethyl-naphtho[2,3-b]furan-4,9-dione, respectively (see scheme 1).

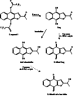

Scheme 1: Compound I is degraded by esterases into active drug, which is further metabolized into active metabolite. During pre-treatment for HPLC analysis, active drug and metabolite are oxidized by oxygen from air to oxidized drug and oxidized metabolite, respectively.

The Waters HPLC system used in this study includes: 1525 dual pump, 2998 photodiode array detector, 2707 auto sampler, Breeze 2 control and analysis software. The HPLC column used in this study: Phenomenex Luna C18, 250×4.60 mm 5 micron. The HPLC mobile phase: buffer A, 80% water, 20% acetonitrile; buffer B, 10% water, 70% acetonitrile, 20% tetrahydrofuran. The HPLC mobile phase gradient: 0-3 min, 50% buffer B to 50% buffer B, 3-10 min, 50% buffer B to 100% buffer B, 10-20 min, 100% buffer B to 100% buffer B, 20-22 min, 100% buffer B−50% buffer B, 22-25 min, 50% buffer B to 50% buffer B; flow rate, 1 ml/min.

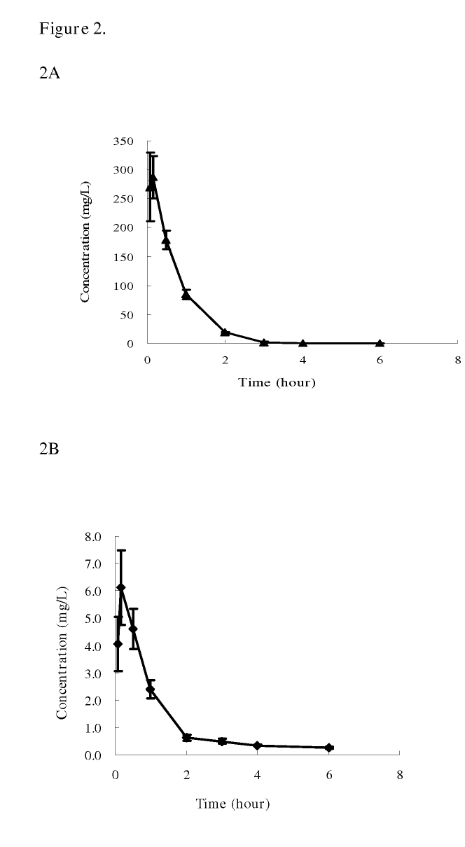

Parameter results of the pharmacokinetic study are shown below. Time courses of compound I, oxidized drug and oxidized active metabolite are illustrated in FIG. 2.

| | | | 2-acetyl- | 2-(1-hydroxy)ethyl- |
|---|---|---|---|---|
| Major | | Com- | naphtho[2,3- | naphtho[2,3- |
| parameters | Units | pound I | b]furan-4,9-dione | b]furan-4,9-dione |
| AUC(0-t) | mg/L*h | 254.686 | 7.417 | 3.801 |
| AUC(0-∞) | mg/L*h | 254.694 | 8.529 | 3.999 |
| t1/2z | h | 0.396 | 3.073 | 0.88 |
| Tmax | h | 0.167 | 0.167 | 0.5 |
| Vz | L/kg | 0.067 | 15.597 | 19.051 |
| CLz | L/h/kg | 0.118 | 3.517 | 15.004 |
| Cmax | mg/L | 287.735 | 6.128 | 1.695 |
| MRT(0-t) | h | 0.628 | 1.248 | 1.438 |
| MRT(0-∞) | h | 0.629 | 2.446 | 1.628 |

### Example 14

### Compound I Anticancer Efficacy Study on Nude Mouse Xenograft Model

HCT116 tumor cells were maintained in vitro as a monolayer culture in DMEM medium supplemented with 10% heat inactivated fetal bovine serum, 100 U/ml penicillin, 100 μg/ml streptomycin, and L-glutamine (2 mM) at 37° C. in an atmosphere of 5% CO₂ in air. The tumor cells were routinely subcultured twice weekly by trypsin-EDTA treatment. The cells growing in an exponential growth phase were harvested and counted for tumor inoculation.

Balb/c nude mice, female, weighing approximately 20±1 grams, were purchased from Shanghai Slac Laboratory Animal Center. Each mouse was inoculated subcutaneously at the right flank with HCT116 tumor cells (10×10⁶) in 0.1 ml of PBS for tumor development. The treatment was started when the mean tumor size reaches approximately 100 mm³. There were three arm groups (6 mice per group) as shown in the following table:

| Group | Mouse | | Dose | |
|---|---|---|---|---|
| Name | number | Test Article | Schedule | Dosage |
| Control | 6 | 2.5% (w/v) poloxamer 188 | iv, bid | 10 ml/kg, |
| | | in saline/PEG400 (5:1) | | 0 mg/kg |
| | | mixture solvent | | Compound I |
| Low | 6 | NS-I-2 prepared according | iv, bid | 10 ml/kg, |
| Dosage | | to the method as described | | 20 mg/kg |
| | | in example 5 | | Compound I |
| High | 6 | NS-I-4 prepared according | iv, bid | 10 ml/kg, |
| Dosage | | to the method as described | | 40 mg/kg |
| | | in example 5 | | Compound I |

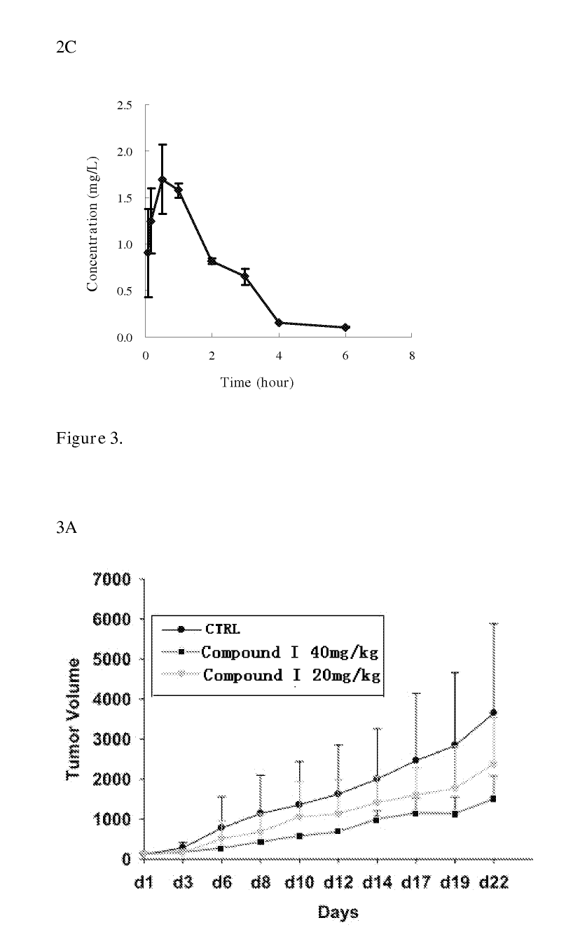

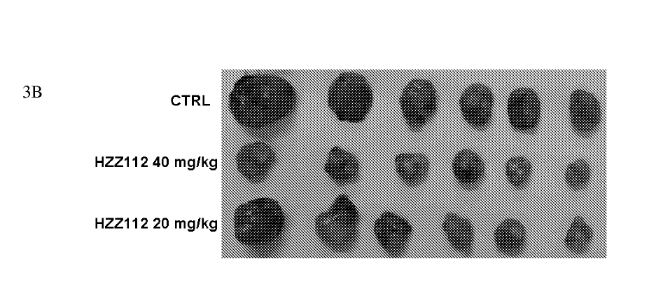

Tumor sizes were measured twice weekly in two dimensions using a caliper, and the volume was expressed in mm³ using the formula: V=0.5 a×b² where a and b were the long and short diameters of the tumor, respectively. The tumor sizes were then used for plotting of tumor volume (mm³) vs time (day) (see FIG. 3A). After had dosed for 22 days, all three group mice were sacrificed and the borne tumors were isolated, weighed, and pictured (see FIG. 3B).

1. A method for preparing an aqueous nanoparticle suspension of a compound of formula I, the method comprising: (1) dissolving a compound of formula I and optionally pharmaceutically acceptable surfactant(s) in a water-miscible organic solvent to form an organic solution; (2) dissolving optionally pharmaceutically acceptable agent(s) and/or optionally pharmaceutically acceptable surfactant(s) in water to form an aqueous solution; and (3) mixing the organic solution and the aqueous solution to form a nanoparticle suspension in which particles have a median particle size (D50) in a range from about 10 nm to about 5000 nm; wherein the compound of formula I is: 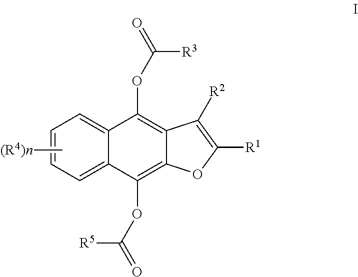
or a pharmaceutically acceptable salt thereof;
wherein:
n is 0-4;
R¹ is independently halogen; —NO₂; —CN; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C₁₋₁₂ aliphatic, 3- to 14-membered carbocyclyl; 3- to 14-membered heterocyclyl; 6- to 14-membered aryl; or 5- to 14-membered heteroaryl;
R² is independently hydrogen; halogen; —NO₂; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C₁₋₁₂ aliphatic; 3- to 14-membered carbocyclyl; 3- to 14-membered heterocyclyl; 6- to 14-membered aryl; or 5- to 14-membered heteroaryl; or
R¹ and R² are taken together with their intervening atoms to form an optionally substituted ring selected from 3- to 14-membered carbocycle, 3- to 14-membered heterocycle, 6- to 14-membered aryl, or 5- to 14-membered heteroaryl;
each R³ and R⁵ is independently an optionally substituted group selected from C₁₋₂₁ aliphatic;
each R⁴ is independently halogen; —NO₂; —CN; —OR; —SR; —N⁺(R)₃; —N(R)₂; —C(O)R; —CO₂R; —C(O)C(O)R; —C(O)CH₂C(O)R; —S(O)R; —S(O)₂R; —C(O)N(R)₂; —SO₂N(R)₂; —OC(O)R; —N(R)C(O)R; —N(R)N(R)₂; —N(R)C(═NR)N(R)₂; —C(═NR)N(R)₂; —C═NOR; —N(R)C(O)N(R)₂; —N(R)SO₂N(R)₂; —N(R)SO₂R; —OC(O)N(R)₂; or an optionally substituted group selected from C₁₋₁₂ aliphatic; 3- to 14-membered carbocyclyl; 3- to 14-membered heterocyclyl; 6- to 14-membered aryl; or 5- to 14-membered heteroaryl, or:
two R⁴ groups on adjacent carbon atoms are taken together with their intervening atoms to form an optionally substituted ring selected from 3- to 14-membered carbocycle; 3- to 14-membered heterocycle; a 6- to 14-membered aryl ring; or a 5- to 14-membered heteroaryl ring;
each R is independently hydrogen or an optionally substituted group selected from C₁₋₁₂ aliphatic; 3- to 14-membered carbocyclyl; 3- to 14-membered heterocyclyl; a 6- to 14-membered aryl; or 5- to 14-membered heteroaryl.

2. The method of claim 1 wherein the compound of formula I is a derivative of 4,9-dihydroxy-naphtho[2,3-b]furan aliphatic acid ester.

3. The method of claim 1 wherein the compound of formula I is selected from the group consisting of 2-acetyl-4,9-dihydroxy-naphtho[2,3-b]furan octanoic acid ester, 2-acetyl-4,9-dihydroxy-naphtho[2,3-b]furan dodecanoic acid ester, and 2-acetyl-4,9-dihydroxy-naphtho[2,3-b]furan hexanoic acid ester.

4. The method of claim 1, wherein the optionally pharmaceutically acceptable surfactant in the water-miscible organic solvent is selected from glycerol mono-(or di-)fatty acid esters, lecithin, phospholipids, cholesterol, PEG-phospholipids, PEG-cholesterol, PEG-cholesterol derivatives, PEG-vitamin A, PEG-vitamin E, PEG-glycerol mono-(or di-)fatty acid esters, ethylene glycol mono-fatty acid esters, propylene glycol mono-fatty acid esters, 3-dialkyl(C1-8)amino-propylene glycol di-fatty acid esters, poly(ethylene glycol) mono-fatty acid esters, stearic acid, sorbitan esters, polyoxyethylene alkyl ethers, polyoxyethylene castor oil derivatives, polyoxyethylene sorbitan fatty acid esters, polyvinyl alcohol, polyvinylpyrrolidone, poloxamers, mixtures of sucrose stearate and sucrose distearate, random copolymers of vinyl acetate and vinyl pyrrolidone, deoxycholic acid, glycodeoxycholic acid, taurocholic acid, or mixtures thereof.

5. The method of claim 1, wherein the optionally pharmaceutically acceptable surfactant in the water-miscible organic solvent includes PEG-phospholipids, PEG-cholesterol, PEG-cholesterol derivatives, PEG-vitamin A, PEG-vitamin E, PEG-glycerol mono-(or di-)fatty acid esters, poly(ethylene glycol) mono-fatty acid esters, polyoxyethylene alkyl ethers, polyoxyethylene castor oil derivatives, polyoxyethylene sorbitan fatty acid esters, poloxamers, and mixtures thereof.

6. The method of claim 1, wherein the water-miscible organic solvent includes ethanol, N-methyl-2-pyrrolidinone, 2-pyrrolidone, dimethyl sulfoxide, dimethylacetamide, polyethylene glycol, propylene glycol, and mixtures thereof.

7. The method of claim 1, wherein the water-miscible organic solvent includes dimethylacetamide, PEG 300, PEG 400, PEG 4000, PEG 20000, and mixtures thereof.

8. The method of claim 1, wherein the water-miscible organic solvent solution contains 0.1-200 mg/ml of a compound of formula I and 0-500 mg/ml of the pharmaceutically acceptable surfactant(s).

9. The method of claim 1, wherein the water-miscible organic solvent solution contains 3-50 mg/ml of a compound of formula I and 0-100 mg/ml of the pharmaceutically acceptable surfactant(s).

10. The method of claim 1, wherein the optionally pharmaceutically acceptable agent in the aqueous solution is selected from mannitol, lactose, maltitol, maltodextrin, maltose, dextrates, dextrin, dextrose, fructose, sorbitol, glucose, sucrose, gelatin, alginic acid and its salt, sodium benzoate, sodium chloride, or mixtures thereof.

11. The method of claim 1, wherein the optionally pharmaceutically acceptable surfactant in the aqueous solution is selected from PEG-phospholipids, PEG-cholesterol, PEG-cholesterol derivatives, PEG-vitamin A, PEG-vitamin E, PEG-glycerol mono-(or di-)fatty acid esters, poly(ethylene glycol) mono-fatty acid esters, polyoxyethylene alkyl ethers, polyoxyethylene castor oil derivatives, polyoxyethylene sorbitan fatty acid esters, albumin, poloxamers, or mixtures thereof.

12. The method of claim 1, wherein the aqueous solution is selected from 0-100 mg/ml albumin in saline, 0-100 mg/ml poloxamer in saline, 0-100 mg/ml of albumin in dextrose solution, or 0-100 mg/ml poloxamer in dextrose solution.

13. The method of claim 1, wherein about 1 to 1000 times volume of the aqueous solution is added into the organic solution of a compound of formula I while stirring or vortexing to form a nanoparticle suspension in which particles have a median particle size (D50) in a range from about 10 nm to about 5000 nm.

14. The method of claim 1, wherein the organic solution of a compound of formula I is added into about 1 to 1000 times volume of the aqueous solution while stirring or vortexing to form a nanoparticle suspension in which particles have a median particle size (D50) in a range from about 10 nm to about 5000 nm.

15. The method of claim 1, wherein the prepared nanoparticle suspension has a median particle size (D50) less than about 5000 nm.

16. The method of claim 1, wherein the prepared nanoparticle suspension has a median particle size (D50) less than about 2000 nm.

17. The method of claim 1, wherein the prepared nanoparticle suspension has a median particle size (D50) less than about 500 nm.

18. The method of claim 1, wherein the prepared nanoparticle suspension has a median particle size (D50) less than about 200 nm.

19. A pharmaceutical composition comprising an aqueous nanoparticle suspension of a compound of formula I, prepared according to the method of claim 1, and optionally a pharmaceutically acceptable carrier.

20-33. (canceled)

34. A kit, which can be used for preparing the pharmaceutical composition of claim 19, comprising:
(a) an organic solution with about 0.1-200 mg/ml of a compound of formula I and 0-500 mg/ml of the pharmaceutically acceptable surfactant(s) in water-miscible organic solvent or ingredients for preparing such organic solution;
(b) an aqueous solution with about 0-200 mg/ml of the pharmaceutically acceptable agent(s) and about 0-200 mg/ml of the pharmaceutically acceptable surfactant(s) or ingredients for preparing such aqueous solution; and
(c) instruction for preparing an aqueous nanoparticle suspension by mixing the organic solution and the aqueous solution, and for preparing the organic solution if ingredients instead of the organic solution are provided in the kit, and also for preparing the aqueous solution if ingredients instead of the aqueous solution are provided in the kit.

35-44. (canceled)

45. A method of treating a subject suffering from or susceptible to a disease, disorder, or condition, the method comprising administering to the subject a therapeutically effective amount of a composition of claim 19 or a composition prepared from a kit of claim 34.

46-58. (canceled)
